(19) United States
(12) Patent Application Publication
(10) Pub. No.: US 2024/0065219 A1
(43) Pub. Date: Feb. 29, 2024

(54) NOVEL LOCI IN GRAPES

(71) Applicant: BLOOM FRESH INTERNATIONAL LIMITED, London (GB)

(72) Inventors: David Cain, Lutz, FL (US); Christopher Owens, Bakersfield, CA (US)

(21) Appl. No.: 18/348,709

(22) Filed: Jul. 7, 2023

Related U.S. Application Data
(60) Provisional application No. 63/368,220, filed on Jul. 12, 2022.

(51) Int. Cl. A01H 6/88; A01H 5/08

(52) U.S. Cl. CPC A01H 6/88; A01H 5/08

(57) ABSTRACT

Table grape plants exhibiting reduced berry browning are provided, together with methods of producing, identifying, or selecting plants or germplasm with a reduced berry browning phenotype. Such plants include table grape plants comprising introgressed genomic regions conferring a reduction in berry browning. Compositions, including novel polymorphic markers for detecting plants comprising introgressed berry browning reduction alleles, are further provided.

### CROSS REFERENCE TO RELATED APPLICATIONS

This application claims the benefit of U.S. Provisional Patent Appl. Ser. No. 63/368,220, filed Jul. 12, 2022, the entire disclosure of which is incorporated herein by reference.

### INCORPORATION OF SEQUENCE LISTING

A sequence listing containing the file named “IFGN018US ST26.xml” which is 20 kilobytes (measured in MS-Windows®) and created on Jun. 30, 2023, and comprises 17 sequences, is incorporated herein by reference in its entirety.

### FIELD OF THE INVENTION

The present invention relates to the field of plant breeding and more specifically to methods and compositions for producing Vitis vinifera plants exhibiting a reduction in berry browning.

### BACKGROUND

Symptoms of berry browning typically include the presence of reddish-brown blotches on the surface of the berry pericarp at the maturing stage and may be classified under one of several different browning phenotypes. The phenomenon of berry browning is multifaceted, and may arise from a combination of factors. These physical blemishes are an undesirable defect that can result in reduced fruit quality and lower prices in the market. Thus, reducing berry browning is particularly important for the commercial production grapes, especially green table grapes. While some management practices have been identified that may reduce berry browning, these practices do not consider the underlying genetics associated with reduced berry browning. Therefore, a continuing need exists in the art to identify alleles conferring a reduction in berry browning as well as more effective methods of introgressing those alleles into commercial lines to provide new varieties with reduced berry browning.

### SUMMARY

In one aspect, provided herein is a Vitis vinifera plant comprising at least a first introgressed allele on a first chromosome selected from the group consisting of chromosomes 5, 9, and 18, and wherein said first introgressed allele confers to said plant a reduction in berry browning compared to a plant not comprising said allele. In some embodiments, said plant further comprises a second introgressed allele on a second chromosome selected from the group consisting of chromosomes 5, 9, and 18, wherein said second introgressed allele confers to a plant a reduction in berry browning compared to a plant not comprising said allele. In particular embodiments, said plant further comprises a third introgressed allele on a third chromosome selected from the group consisting of chromosomes 5, 9, and 18, wherein said third introgressed allele confers to a plant a reduction in berry browning compared to a plant not comprising said allele. In certain embodiments, said first introgressed allele is flanked in the genome of said plant by: marker locus M1 (SEQ ID NO: 1) and marker locus M6 (SEQ ID NO: 6) on chromosome 5; marker locus M7 (SEQ ID NO: 7) and marker locus M11 (SEQ ID NO: 11) on chromosome 9; or marker locus M12 (SEQ ID NO: 12) and marker locus M17 (SEQ ID NO:17) on chromosome 18. In other embodiments, a representative deposit of a plant comprising said first, second, and third alleles has been deposited under NCMA Accession No. 202110015. In yet other embodiments, said first introgressed allele is located at a position in the genome of said plant flanked by marker locus M1 (SEQ ID NO: 1) and marker locus M6 (SEQ ID NO: 6) on chromosome 5 and wherein said allele comprises the haplotype for said allele found in Vitis vinifera variety ‘Valley Pearl’ or ‘Shingargoon’. In still further embodiments, said plant is homozygous for said first introgressed allele; said plant is heterozygous for said first introgressed allele; said plant is homozygous for said second introgressed allele; said plant is heterozygous for said second introgressed allele; said plant is homozygous for said third introgressed allele; said plant is heterozygous for said third introgressed allele; or said first introgressed allele is on chromosome 5 or chromosome 18, and wherein said plant exhibits a reduction in berry browning compared to a plant that homozygous for said first allele. Cells, seeds, and plant parts comprising said at least a first introgressed allele are further provided. In certain embodiments, said plant part is a cell, a seed, a root tip, an ovule, an embryo, or pollen.

In another aspect, provided herein is a method for producing a Vitis vinifera plant with reduced oxidative browning comprising crossing the plant of claim 1 with itself or with a Vitis vinifera plant of a different genotype to produce one or more progeny plants; and selecting a progeny plant comprising said allele that confers reduced oxidative browning. In some embodiments, selecting said progeny plant comprises detecting a marker locus genetically linked to said reduced oxidative browning allele. In other embodiments, selecting said progeny plant comprises detecting a marker locus within or genetically linked to a chromosomal segment flanked in the genome of said plant by: marker locus M1 (SEQ ID NO: 1) and marker locus M6 (SEQ ID NO: 6) on chromosome 5; marker locus M7 (SEQ ID NO: 7) and marker locus M11 (SEQ ID NO: 11) on chromosome 9; or marker locus M12 (SEQ ID NO: 12) and marker locus M17 (SEQ ID NO:17) on chromosome 18. In still other embodiments, selecting a progeny plant comprises detecting nucleic acids comprising a marker locus selected from the group consisting of marker locus M1 (SEQ ID NO: 1), marker locus M2 (SEQ ID NO: 2), marker locus M3 (SEQ ID NO: 3), marker locus M4 (SEQ ID NO: 4), marker locus M5 (SEQ ID NO: 5), marker locus M6 (SEQ ID NO: 6), marker locus M7 (SEQ ID NO: 7), marker locus M8 (SEQ ID NO: 8), marker locus M9 (SEQ ID NO: 9), marker locus M10 (SEQ ID NO: 10), marker locus M11 (SEQ ID NO: 11), marker locus M12 (SEQ ID NO: 12), marker locus M13 (SEQ ID NO: 13), marker locus M14 (SEQ ID NO: 14), marker locus M15 (SEQ ID NO: 15), marker locus M16 (SEQ ID NO: 16), and marker locus M17 (SEQ ID NO: 17), or a marker locus located within 10 cM thereof. In still other embodiments, selecting a progeny plant comprises detecting nucleic acids comprising marker locus M3 (SEQ ID NO: 3) and marker locus M4 (SEQ ID NO: 4) or marker locus M14 (SEQ ID NO: 14) and marker locus M15 (SEQ ID NO: 15). In particular embodiments, selecting said progeny plant comprises identifying a genetic marker within or genetically linked to a genomic region between marker locus M7 (SEQ ID NO: 7) and marker locus M11 (SEQ ID NO: 11) on chromosome 9. In yet further embodiments, selecting said progeny plant further comprises detecting at least one polymorphism at a locus selected from the group consisting of marker locus marker locus M8 (SEQ ID NO: 8), M9 (SEQ ID NO: 9), and marker locus M10 (SEQ ID NO: 10). In still other embodiments, the progeny plant is an F₂-F₆ progeny plant or producing the progeny plant comprises backcrossing.

In another aspect, provided herein is a method of producing a plant of a table grape variety exhibiting reduced berry browning, comprising introgressing into a plant a reduced oxidative browning allele, wherein said reduced oxidative browning allele is defined as located in a genomic region between: marker locus M1 (SEQ ID NO: 1) and marker locus M6 (SEQ ID NO: 6) on chromosome 5; marker locus M7 (SEQ ID NO: 7) and marker locus M11 (SEQ ID NO: 11) on chromosome 9; or marker locus M12 (SEQ ID NO: 12) and marker locus M17 (SEQ ID NO:17) on chromosome 18. In some embodiments, said introgressing comprises backcrossing or marker-assisted selection. In other embodiments, said introgressing comprises detecting nucleic acids comprising marker locus M1 (SEQ ID NO: 1), marker locus M2 (SEQ ID NO: 2), marker locus M3 (SEQ ID NO: 3), marker locus M4 (SEQ ID NO: 4), marker locus M5 (SEQ ID NO: 5), marker locus M6 (SEQ ID NO: 6), marker locus M7 (SEQ ID NO: 7), marker locus M8 (SEQ ID NO: 8), marker locus M9 (SEQ ID NO: 9), marker locus M10 (SEQ ID NO: 10), marker locus M11 (SEQ ID NO: 11), marker locus M12 (SEQ ID NO: 12), marker locus M13 (SEQ ID NO: 13), marker locus M14 (SEQ ID NO: 14), marker locus M15 (SEQ ID NO: 15), marker locus M16 (SEQ ID NO: 16), or marker locus M17 (SEQ ID NO: 17).

In yet another aspect, methods are provided for selecting a plant of a table grape variety exhibiting reduced berry browning, comprising: obtaining a population of progeny plants having at least one parent exhibiting reduced berry browning; screening said population with at least one nucleic acid marker to detect a locus associated with an allele that confers reduced oxidative browning; and selecting from said population one or more progeny plants based on the presence of said locus, wherein said locus is within or genetically linked to a chromosomal segment flanked in the genome of said plant by: marker locus M1 (SEQ ID NO: 1) and marker locus M6 (SEQ ID NO: 6) on chromosome 5; marker locus M7 (SEQ ID NO: 7) and marker locus M11 (SEQ ID NO: 11) on chromosome 9; or marker locus M12 (SEQ ID NO: 12) and marker locus M17 (SEQ ID NO:17) on chromosome 18. In some embodiments, selecting said progeny plants comprises detecting nucleic acids comprising a marker locus selected from the group consisting of marker locus M1 (SEQ ID NO: 1), marker locus M2 (SEQ ID NO: 2), marker locus M3 (SEQ ID NO: 3), marker locus M4 (SEQ ID NO: 4), marker locus M5 (SEQ ID NO: 5), marker locus M6 (SEQ ID NO: 6), marker locus M7 (SEQ ID NO: 7), marker locus M8 (SEQ ID NO: 8), marker locus M9 (SEQ ID NO: 9), marker locus M10 (SEQ ID NO: 10), marker locus M11 (SEQ ID NO: 11), marker locus M12 (SEQ ID NO: 12), marker locus M13 (SEQ ID NO: 13), marker locus M14 (SEQ ID NO: 14), marker locus M15 (SEQ ID NO: 15), marker locus M16 (SEQ ID NO: 16), and marker locus M17 (SEQ ID NO: 17), or a marker locus located within 10 cM thereof. In certain embodiments, said progeny plant is an F2-F6 progeny plant. In other embodiments, screening said population comprises PCR, single strand conformational polymorphism analysis, denaturing gradient gel electrophoresis, cleavage fragment length polymorphism analysis, TAQMAN assay, and/or DNA sequencing.

In a further aspect, methods are provided for identifying a Vitis vinifera plant comprising a reduced oxidative browning allele: obtaining nucleic acids from at least a first Vitis vinifera plant; and identifying in said nucleic acids the presence of at least a first genetic marker indicative of the presence of a chromosomal segment flanked in the genome of said plant by: marker locus M1 (SEQ ID NO: 1) and marker locus M6 (SEQ ID NO: 6) on chromosome 5; marker locus M7 (SEQ ID NO: 7) and marker locus M11 (SEQ ID NO: 11) on chromosome 9; or marker locus M12 (SEQ ID NO: 12) and marker locus M17 (SEQ ID NO:17) on chromosome 18, wherein said reduced oxidative browning allele confers to said plant reduced berry browning compared to a plant not comprising said allele. In some embodiments, said identifying comprises detecting a marker genetically linked to marker locus M2 (SEQ ID NO: 2), marker locus M3 (SEQ ID NO: 3), marker locus M4 (SEQ ID NO: 4), and marker locus M5 (SEQ ID NO: 5); marker locus M8 (SEQ ID NO: 8), marker locus M9 (SEQ ID NO: 9), and marker locus M10 (SEQ ID NO: 10); or marker locus M13 (SEQ ID NO: 13), marker locus M14 (SEQ ID NO: 14), marker locus M15 (SEQ ID NO: 15), and marker locus M16 (SEQ ID NO: 16). In certain embodiments, the Vitis vinifera plant is a plant embryo. The present invention further provides Vitis vinifera plants obtainable by the methods provided herein. Also provided herein is a plant that is a progeny plant of any generation of a plant according to the invention, wherein the plant comprises at least a first introgressed allele on a first chromosome as described herein selected from the group consisting of chromosomes 5, 9, and 18, and wherein said first introgressed allele confers to said plant a reduction in berry browning compared to a plant not comprising said allele.

In yet a further aspect, provided herein is a interspecific hybrid Vitis plant produced from a cross between a Vitis vinifera plant and a plant of a distinct Vitis species, or a progeny thereof, comprising at least a first introgressed allele on a first chromosome selected from the group consisting of chromosomes 5, 9, and 18, and wherein said first introgressed allele confers to said plant a reduction in berry browning compared to a plant not comprising said allele. In certain embodiments, embryos that produce said interspecific hybrid Vitis plant are also provided.

### BRIEF DESCRIPTION OF THE DRAWINGS

FIG. 1: Shows a Manhattan plot corresponding to the GWAS analysis using an additive genetic model. The significance threshold is represented by the dashed lines. The analysis identified 2 isolated QTLs in chromosomes 9 and 18.

FIG. 2: Shows a Manhattan plot corresponding to the GWAS analysis using a dominance genetic model. The significance threshold is represented by the dashed lines. The analysis identified an isolated QTL in chromosomes 5.

FIG. 3: Shows a Boxplot of observed berry browning as a function of allele substitution at position 17,373,984 on chromosome 5. On the y-axis, 100% represents no berry browning and 0% representing complete berry browning. On the x-axis, plants homozygous “CC”; plants heterozygous “GC”; and plants homozygous “GG” at the allele are represented (left to right).

FIG. 4: Shows a Boxplot of observed berry browning as a function of allele substitution at position 17,600,454 on chromosome 9. On the y-axis, 100% represents no berry browning and 0% representing complete berry browning. On the x-axis, plants homozygous “CC”; plants heterozygous “CT”; and plants homozygous “TT” at the allele are represented (left to right).

FIG. 5: Shows a Boxplot of observed berry browning as a function of allele substitution at position 27,007,362 on chromosome 18. On the y-axis, 100% represents no berry browning and 0% representing complete berry browning. On the x-axis, plants homozygous “AA”; plants heterozygous “AC”; and plants homozygous “CC” at the allele are represented (left to right).

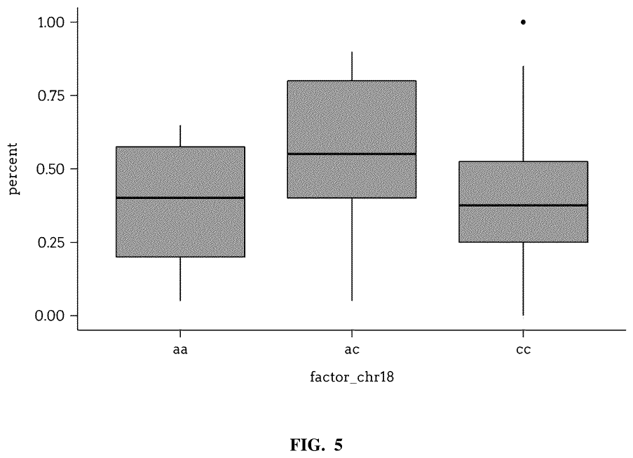

### DETAILED DESCRIPTION

The value of table grapes depends on their visual appearance. Berry browning, which occurs at the maturation stage just before harvest can greatly diminish the value of table grapes. This phenomenon was first reported in 1989, and several different classifications of browning (e.g. external, internal, low temperature, chemical, physical, and pathogenic browning) have been described to date. Several management approaches to either delay or reduce this problem have been developed. These management approaches include harvesting varieties known to be prone to browning early, treating vines with plant growth regulators to delay berry ripening, adjusting bunches to hang freely, and removing leaves around bunches to prevent rubbing. Previous studies have also shown that dysfunction or disruption of cellular membranes, which allows the mixing of the polyphenol oxidase enzymes (PPO) with phenolic substrates occurring naturally in the fruit, contributes to berry browning. However, genetic loci associated with a reduction in berry browning in table grapes are not known in the art, severely limiting the ability of plant breeder's to sustainably reduce berry browning.

The present disclosure provides genome wide association studies (GWAS) using a large Vitis vinifera panel in order to assess the genetic architecture of berry browning resistance. Through the GWAS (MLMM) approach described herein, three loci conferring a reduction in berry browning were identified on chromosome 5 (“chr5”), chromosome 9 (“chr9”), and chromosome 18 (“chr18”). The loci on chromosomes 5, 9, and 18 described herein represent newly identified alleles associated with a reduction in berry browning and provide a valuable new tool for reducing berry browning in table grapes. Thus, the present invention represents a significant advance in that it provides, in one embodiment, berry browning reduction in table grape plants conferred by novel QTLs on chromosomes 5, 9, and 18. In addition, novel markers for the new loci are provided, allowing each locus to be accurately introgressed and tracked during development of new varieties of table grapes, including interspecific hybrid Vitis varieties. As such, the invention permits introgression of the berry browning reduction loci into potentially any desired table grape genotype.

The present inventors have discovered for the first time that, M1, a SNP marker with a [G/A] change at 17,371,597 bp on chromosome 5 of the public Vitis vinifera genome map (www.genoscope.cns.fr/externe/GenomeBrowser/Vitis/#:˜:text=This %20Grapevine %20genome %20sequence %20analysis, and %20the %20University %20 of %20Verona.), M6, a SNP marker with a [C/T] change at 17,401,109 bp on chromosome 5 of the public Vitis vinifera genome map, M7, a SNP marker with a [A/C] change at 17,551,003 bp on chromosome 9 of the public Vitis vinifera genome map, M11, a SNP marker with a [A/G] change at 17,600,713 bp on chromosome 9 of the public Vitis vinifera genome map, M12, a SNP marker with a [A/T] change at 27,001,788 bp on chromosome 18 of the public Vitis vinifera genome map, and M17, a SNP marker with a [A/G] change at 27,007,611 bp on chromosome 18 of the public Vitis vinifera genome map can be used to identify these regions, wherein M1 and M6 are flanking markers for the chromosomal segment on chromosome 5; wherein M7 and M11 are flanking markers for the chromosomal segment on chromosome 9; and wherein M12 and M17 are flanking markers for the chromosomal segment on chromosome 18. The public genome of V. vinifera is available at, e.g. www.genoscope.cns.fr/externe/GenomeBrowser/Vitis/#:˜:text=This %20Grapevine %20genome %20sequence %20analysis, and %20the %20University %20 of %20Verona, and one skilled in the art would understand that the marker sequences provided for the first time in the instant application could be located on any version (or later version) of the public genome.

In some embodiments, the invention provides plants comprising at least a first introgressed allele on a first chromosome selected from the group consisting of chromosomes 5, 9, and 18, and wherein said first introgressed allele confers to said plant a reduction in berry browning compared to a plant not comprising said allele, such as a plant of the same variety grown under the same conditions but lacking the introgression. Methods of producing the plants described herein are further provided. The invention further provides novel trait-linked markers which can be used to produce plants comprising the recombinant introgression, including the markers shown in Table 1, and markers M3 (SEQ ID NO: 3), M4 (SEQ ID NO: 4), M9 (SEQ ID NO: 9), M14 (SEQ ID NO: 14), and M15 (SEQ ID NO: 15), which have been shown to be genetically linked to berry browning reduction in table grape plants.

In other embodiments, provided herein are methods of selecting a plant of a table grape variety exhibiting reduced berry browning. In other embodiments, said methods comprise screening one or more plants with at least one nucleic acid marker to detect a locus associated with an allele that confers reduced oxidative browning. In certain embodiments, said method comprises selecting a plant based on the presence of said locus, wherein said locus is within or genetically linked to a chromosomal segment flanked in the genome of said plant by marker locus M1 (SEQ ID NO: 1) and marker locus M6 (SEQ ID NO: 6) on chromosome 5; marker locus M7 (SEQ ID NO: 7) and marker locus M11 (SEQ ID NO: 11) on chromosome 9; or marker locus M12 (SEQ ID NO: 12) and marker locus M17 (SEQ ID NO:17) on chromosome 18. In particular embodiments, selecting a table grape variety exhibiting a reduction in berry browning comprises molecular genetic techniques. For example, those of ordinary skill in the art viewing the present disclosure may use technical methods to select a table grape plant exhibiting reduced berry browning by screening one or more plants with at least one nucleic acid marker to detect a polymorphism genetically linked to reduced berry browning resistance.

In certain embodiments, the invention provides plants comprising one or more of the novel alleles provided herein. These novel alleles provide a reduction in berry browning. Methods of producing the plants described herein are further provided. In certain embodiments, the invention provides V. vinifera line ‘04009-039-230’ comprising the alleles on chromosomes 5, 9, and 18, as described herein, a representative sample of plant tissue having been deposited under NCMA Accession No. 202110015.

In particular embodiments, interspecific hybrid Vitis plants comprising the berry browning reduction alleles described herein are also provided. One example of such an interspecific hybrid Vitis plant includes a plant produced from a cross between a Vitis vinifera plant and a plant of a distinct Vitis species, or a progeny thereof, comprising at least a first introgressed allele on a first chromosome selected from the group consisting of chromosomes 5, 9, and 18, and wherein said first introgressed allele confers to said plant a reduction in berry browning compared to a plant not comprising said allele. Distinct Vitis species include, but are not limited to, Vitis labrusca, cinerea, aestivalis, riparia, rupestris, romanetti, piasezkii, mustangensis, monticola, girdiana, arizonica, rotundifolia, and acerifolia. One of skill in the art would therefore understand that the alleles, polymorphisms, and markers provided by the present disclosure allow for identifying, selecting, tracking, and introducing any of the genomic regions identified herein into any genetic background, including interspecific hybrid Vitis plants.

A technique which has been used to obtain viable interspecific hybrid Vitis plants is embryo rescue (Dolezel el al. 1980). Thus, in some embodiments the invention provides a process of producing an interspecific hybrid Vitis plant or part thereof. In one embodiment, the process comprises crossing a Vitis vinifera plant and a plant of a distinct Vitis species. The interspecific hybrid Vitis plant comprising at least a first introgressed allele on a first chromosome selected from the group consisting of chromosomes 5, 9, and 18, and wherein said first introgressed allele confers to said plant a reduction in berry browning compared to a plant not comprising said allele. The process may still further be defined as comprising the steps of (a) cultivating first and second plants, wherein either the first plant is a Vitis vinifera plant and the second plant is a plant of a distinct Vitis species, or the first plant is a plant of a distinct Vitis species and the second plant is a Vitis vinifera plant; (b) collecting pollen from the first plant, (c) pollinating a flower on the second plant with the pollen; and (d) obtaining an interspecific hybrid plant resulting from the pollinating. The process may still further be defined as comprising embryo rescue of an embryo resulting from the pollinating. In particular embodiments of the invention, the first plant is a Vitis vinifera plant and the second plant is a plant of a distinct Vitis species, while in other embodiments the first plant is a plant of a distinct Vitis species and the second plant is a Vitis vinifera plant. In yet another aspect, the invention provides a plant or any part thereof produced by crossing a Vitis vinifera plant to a plant of a distinct Vitis species, including any clonal propagations thereof, wherein the interspecific hybrid Vitis plant comprising at least a first introgressed allele on a first chromosome selected from the group consisting of chromosomes 5, 9, and 18, and wherein said first introgressed allele confers to said plant a reduction in berry browning compared to a plant not comprising said allele.

### I. Genomic Regions, Alleles, and Polymorphisms Associated with Berry Browning Reduction in Grape Plants

The invention provides novel introgressions of one or more alleles associated with a reduction in berry browning of table grape plants, together with polymorphic nucleic acids and markers for tracking the introgressions during plant breeding.

V. vinifera lines exhibiting a reduction in berry browning are described herein and may be used together with the trait-linked markers provided herein in accordance with the present disclosure. For example, V. vinifera line ‘04009-039-230’ comprising the chromosomal segments as described herein, a representative sample of plant tissue having been deposited under NCMA Accession No. 202110015, can be used as a source for the berry browning reduction alleles according to the disclosure. Additionally, V. vinifera line ‘Valley Pearl’ (US PP23,422), originally created by the USDA, and V. vinifera line ‘Shingargoon’, which is available through the USDA (npgsweb.ars-grin.gov/gringlobal/accessiondetail?id=1564481), both comprise a favorable allele at a position in the genome of flanked by marker locus M1 (SEQ ID NO: 1) and marker locus M6 (SEQ ID NO: 6) on chromosome 5. Thus, each of these publicly available V. vinifera lines represents a source for a favorable haplotype for conferring berry browning reduction according to the disclosure.

Using the improved genetic markers and the assays described herein, those skilled in the art are able to successfully produce and/or select plants comprising the berry browning reduction alleles described herein, which confer increased reduction to berry browning as compared to a plant not comprising the allele(s). In certain embodiments, provided herein are methods of introgressing into a plant an allele conferring reduced berry browning within a chromosomal segment flanked in the genome of the plant by marker locus M1 (SEQ ID NO:1) and marker locus M6 (SEQ ID NO:6) on chromosome 5; marker locus M7 (SEQ ID NO:7) and marker locus M11 (SEQ ID NO:11) on chromosome 9; and/or marker locus M12 (SEQ ID NO:12) and marker locus M17 (SEQ ID NO:17) on chromosome 18. The present disclosure therefore represents a significant advance in the art.

### II. Introgression of Genomic Regions Associated with Berry Browning Reduction in Grape Plants

Marker-assisted introgression involves the transfer of a chromosomal region defined by one or more markers from a first genetic background to a second. Offspring of a cross that contain the introgressed genomic region can be identified by the combination of markers characteristic of the desired introgressed genomic region from a first genetic background and both linked and unlinked markers characteristic of the second genetic background.

Provided herein are accurate markers for identifying and tracking introgression of one or more of the genomic regions disclosed herein from a plant exhibiting reduced berry browning into a cultivated line. Further provided are markers for identifying and tracking the introgressions disclosed herein during plant breeding, including the markers set forth in Table 1.

Markers within or linked to any of the genomic intervals described herein may be useful in a variety of breeding efforts that include introgression of genomic regions associated with disease resistance into a desired genetic background. For example, a marker within 40 cM, 20 cM, 15 cM, 10 cM, 5 cM, 2 cM, or 1 cM of a marker associated with disease resistance described herein can be used for marker-assisted introgression of genomic regions associated with a disease resistant phenotype.

Grape plants comprising one or more introgressed regions associated with a desired phenotype wherein at least 10%, 25%, 50%, 75%, 90%, or 99% of the remaining genomic sequences carry markers characteristic of the recurrent parent germplasm are also provided. Grape plants comprising an introgressed region comprising regions closely linked to or adjacent to the genomic regions and markers provided herein and associated with a resistance phenotype are also provided.

### III. Development of Grape Varieties Exhibiting a Reduction in Berry Browning

For most breeding objectives, commercial breeders work within germplasm that is “cultivated,” “cultivated type,” or “elite.” These cultivated lines may be used as recurrent parents or as a source of recurrent parent alleles during breeding. Cultivated or elite germplasm is easier to breed because it generally performs well when evaluated for horticultural performance. Many cultivated grape types have been developed and are known in the art as being agronomically elite and appropriate for commercial cultivation. However, the performance advantage a cultivated germplasm provides can be offset by a lack of allelic diversity. Breeders generally accept this tradeoff because progress is faster when working with cultivated material than when breeding with genetically diverse sources.

In contrast, when cultivated germplasm is crossed with non-cultivated germplasm, a breeder can gain access to novel alleles from the non-cultivated type. Non-cultivated germplasm may be used as a source of donor alleles during breeding. However, this approach generally presents significant difficulties due to fertility problems associated with crosses between diverse lines, and negative linkage drag from the non-cultivated parent. For example, non-cultivated grape types can provide alleles associated with disease resistance. However, these non-cultivated types may have poor horticultural qualities such as poor fruit shape, agronomically unacceptable plant architecture, and/or small fruit size.

The process of introgressing desirable resistance genes from non-cultivated lines into elite cultivated lines while avoiding problems with linkage drag or low heritability is a long and often arduous process. In deploying alleles derived from wild relatives it is often desirable to introduce a minimal or truncated introgression that provides the desired trait but lacks detrimental effects. To aid introgression reliable marker assays are preferable to phenotypic screens. Success is furthered by simplifying genetics for key attributes to allow focus on genetic gain for quantitative traits such as reduced berry browning or disease resistance. Moreover, the process of introgressing genomic regions from non-cultivated lines can be greatly facilitated by the availability of accurate markers for MAS.

One of skill in the art would therefore understand that the alleles, polymorphisms, and markers provided by the present disclosure allow the tracking and introduction of any of the genomic regions identified herein into any genetic background, including interspecific hybrid Vitis plants. In addition, the genomic regions associated with reduced berry browning disclosed herein can be introgressed from one genotype to another and tracked using MAS. Thus, the disclosure of accurate markers associated with a reduction in berry browning will facilitate the development of grape plants having beneficial phenotypes. For example, plants or parts thereof can be genotyped using the markers of the present disclosure to select for plants comprising desired genomic regions associated with reduced berry browning. Moreover, MAS allows identification of plants homozygous or heterozygous for a desired introgression.

Inter-species crosses can also result in suppressed recombination and plants with low fertility or fecundity. For example, suppressed recombination has been observed for the tomato nematode resistance gene Mi, the Mla and Mlg genes in barley, the Yr17 and Lr20 genes in wheat, the RunI gene in grapevine, and the Rma gene in peanut. Meiotic recombination is essential for classical breeding because it enables the transfer of favorable alleles across genetic backgrounds, the removal of deleterious genomic fragments, and pyramiding traits that are genetically tightly linked. Therefore suppressed recombination forces breeders to enlarge segregating populations for progeny screens in order to arrive at the desired genetic combination.

Phenotypic evaluation of large populations is time-consuming, resource-intensive and not reproducible in every environment. Marker-assisted selection offers a feasible alternative. Molecular assays designed to detect unique polymorphisms, such as SNPs, are versatile. However, they may fail to discriminate alleles within and among grape species in a single assay. Structural rearrangements of chromosomes such as deletions impair hybridization and extension of synthetically labeled oligonucleotides. In the case of duplication events, multiple copies are amplified in a single reaction without distinction. The development and validation of accurate and highly predictive markers are therefore essential for successful MAS breeding programs.

### IV. Marker Assisted Breeding and Genetic Engineering Techniques

Potentially any type of genetic marker may find use with the invention, as described herein. Non-limiting examples of such markers include restriction fragment length polymorphisms (RFLPs), amplified fragment length polymorphisms (AFLPs), simple sequence repeats (SSRs), simple sequence length polymorphisms (SSLPs), single nucleotide polymorphisms (SNPs), insertion/deletion polymorphisms (Indels), variable number tandem repeats (VNTRs), and random amplified polymorphic DNA (RAPD), isozymes, and other markers known to those skilled in the art. Marker discovery and development in crop plants provides the initial framework for applications to marker-assisted breeding activities (U.S. Patent Pub. Nos.: 2005/0204780, 2005/0216545, 2005/0218305, and 2006/00504538). The resulting “genetic map” is the representation of the relative position of characterized loci (polymorphic nucleic acid markers or any other locus for which alleles can be identified) to each other.

Polymorphisms comprising as little as a single nucleotide change can be assayed in a number of ways. For example, detection can be made by electrophoretic techniques including a single strand conformational polymorphism (Orita, et al. (1989) Genomics, 8(2), 271-278), denaturing gradient gel electrophoresis (Myers (1985) EPO 0273085), or cleavage fragment length polymorphisms (Life Technologies, Inc., Gaithersburg, MD), but the widespread availability of DNA sequencing often makes it easier to simply sequence amplified products directly. Once the polymorphic sequence difference is known, rapid assays can be designed for progeny testing, typically involving some version of PCR amplification of specific alleles (PASA; Sommer, et al. (1992) Biotechniques 12(1), 82-87), or PCR amplification of multiple specific alleles (PAMSA; Dutton and Sommer (1991) Biotechniques, 11(6), 700-7002).

Polymorphic markers serve as useful tools for assaying plants for determining the degree of identity of lines or varieties (U.S. Pat. No. 6,207,367). These markers form the basis for determining associations with phenotypes and can be used to drive genetic gain. In certain embodiments, polymorphic nucleic acids can be used to detect in a grape plant a genotype associated with disease resistance, identify a grape plant with a genotype associated with disease resistance, and to select a grape plant with a genotype associated with disease resistance. In certain embodiments of methods described, polymorphic nucleic acids can be used to produce a grape plant that comprises in its genome an introgressed locus associated with reduced berry browning. In certain embodiments, polymorphic nucleic acids can be used to breed progeny grape plants comprising a locus or loci associated with disease resistance.

Genetic markers may include dominant or codominant markers. Codominant markers reveal the presence of two or more alleles (two per diploid individual). Dominant markers reveal the presence of only a single allele. Markers are preferably inherited in codominant fashion so that the presence of both alleles at a diploid locus, or multiple alleles in triploid or tetraploid loci, are readily detectable, and they are free of environmental variation, i.e., their heritability is 1. A marker genotype typically comprises two marker alleles at each locus in a diploid organism. The marker allelic composition of each locus can be either homozygous or heterozygous. Homozygosity is a condition where both alleles at a locus are characterized by the same nucleotide sequence. Heterozygosity refers to a condition where the two alleles at a locus are different.

Nucleic acid-based analyses for determining the presence or absence of the genetic polymorphism (i.e. for genotyping) can be used in breeding programs for identification, selection, introgression, and the like. A wide variety of genetic markers for the analysis of genetic polymorphisms are available and known to those of skill in the art. The analysis may be used to select for genes, portions of genes, QTL, alleles, or genomic regions that comprise or are linked to a genetic marker that is linked to or associated with a favorable trait (e.g. a reduction in berry browning or increased disease resistance) in grape plants.

As used herein, nucleic acid analysis methods include, but are not limited to, PCR-based detection methods (for example, TaqMan assays), microarray methods, mass spectrometry-based methods and/or nucleic acid sequencing methods, including whole genome sequencing. In certain embodiments, the detection of polymorphic sites in a sample of DNA, RNA, or cDNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis, fluorescence detection methods, or other means.

One method of achieving such amplification employs the polymerase chain reaction (PCR) (Mullis et al. (1986) Cold Spring Harbor Symp. Quant. Biol. 51:263-273; European Patent 50,424; European Patent 84,796; European Patent 258,017; European Patent 237,362; European Patent 201,184; U.S. Pat. Nos. 4,683,202; 4,582,788; and 4,683,194), using primer pairs that are capable of hybridizing to the proximal sequences that define a polymorphism in its double-stranded form. Methods for typing DNA based on mass spectrometry can also be used. Such methods are disclosed in U.S. Pat. Nos. 6,613,509 and 6,503,710, and references found therein.

Polymorphisms in DNA sequences can be detected or typed by a variety of effective methods well known in the art including, but not limited to, those disclosed in U.S. Pat. Nos. 5,468,613, 5,217,863; 5,210,015; 5,876,930; 6,030,787; 6,004,744; 6,013,431; 5,595,890; 5,762,876; 5,945,283; 5,468,613; 6,090,558; 5,800,944; 5,616,464; 7,312,039; 7,238,476; 7,297,485; 7,282,355; 7,270,981 and 7,250,252 all of which are incorporated herein by reference in their entirety. However, the compositions and methods of the present disclosure can be used in conjunction with any polymorphism typing method to detect polymorphisms in genomic DNA samples. These genomic DNA samples used include but are not limited to, genomic DNA isolated directly from a plant, cloned genomic DNA, or amplified genomic DNA.

For instance, polymorphisms in DNA sequences can be detected by hybridization to allele-specific oligonucleotide (ASO) probes as disclosed in U.S. Pat. Nos. 5,468,613 and 5,217,863. U.S. Pat. No. 5,468,613 discloses allele specific oligonucleotide hybridizations where single or multiple nucleotide variations in nucleic acid sequence can be detected in nucleic acids by a process in which the sequence containing the nucleotide variation is amplified, spotted on a membrane and treated with a labeled sequence-specific oligonucleotide probe.

Target nucleic acid sequence can also be detected by probe ligation methods, for example as disclosed in U.S. Pat. No. 5,800,944 where sequence of interest is amplified and hybridized to probes followed by ligation to detect a labeled part of the probe.

Microarrays can also be used for polymorphism detection, wherein oligonucleotide probe sets are assembled in an overlapping fashion to represent a single sequence such that a difference in the target sequence at one point would result in partial probe hybridization (Borevitz et al., Genome Res. 13:513-523 (2003); Cui et al., Bioinformatics 21:3852-3858 (2005). On any one microarray, it is expected there will be a plurality of target sequences, which may represent genes and/or noncoding regions wherein each target sequence is represented by a series of overlapping oligonucleotides, rather than by a single probe. This platform provides for high throughput screening of a plurality of polymorphisms. Typing of target sequences by microarray-based methods is described in U.S. Pat. Nos. 6,799,122; 6,913,879; and 6,996,476.

Other methods for detecting SNPs and Indels include single base extension (SBE) methods. Examples of SBE methods include, but are not limited, to those disclosed in U.S. Pat. Nos. 6,004,744; 6,013,431; 5,595,890; 5,762,876; and 5,945,283.

In another method for detecting polymorphisms, SNPs and Indels can be detected by methods disclosed in U.S. Pat. Nos. 5,210,015; 5,876,930; and 6,030,787 in which an oligonucleotide probe having a 5′ fluorescent reporter dye and a 3′ quencher dye covalently linked to the 5′ and 3′ ends of the probe. When the probe is intact, the proximity of the reporter dye to the quencher dye results in the suppression of the reporter dye fluorescence, e.g. by Forster-type energy transfer. During PCR, forward and reverse primers hybridize to a specific sequence of the target DNA flanking a polymorphism while the hybridization probe hybridizes to polymorphism-containing sequence within the amplified PCR product. In the subsequent PCR cycle DNA polymerase with 5′→3′ exonuclease activity cleaves the probe and separates the reporter dye from the quencher dye resulting in increased fluorescence of the reporter.

In another embodiment, a locus or loci of interest can be directly sequenced using nucleic acid sequencing technologies. Methods for nucleic acid sequencing are known in the art and include technologies provided by 454 Life Sciences (Branford, CT), Agencourt Bioscience (Beverly, MA), Applied Biosystems (Foster City, CA), LI-COR Biosciences (Lincoln, NE), NimbleGen Systems (Madison, WI), Illumina (San Diego, CA), and VisiGen Biotechnologies (Houston, TX). Such nucleic acid sequencing technologies comprise formats such as parallel bead arrays, sequencing by ligation, capillary electrophoresis, electronic microchips, “biochips,” microarrays, parallel microchips, and single-molecule arrays.

Various genetic engineering technologies have been developed and may be used by those of skill in the art to introduce traits in plants. In certain aspects, traits are introduced into grape plants via altering or introducing a single genetic locus or transgene into the genome of a variety or progenitor thereof. Methods of genetic engineering to modify, delete, or insert genes and polynucleotides into the genomic DNA of plants are well-known in the art.

In specific embodiments, improved grape lines can be created through the site-specific modification of a plant genome. Methods of genetic engineering include, for example, utilizing sequence-specific nucleases such as zinc-finger nucleases (see, for example, U.S. Pat. Appl. Pub. No. 2011-0203012); engineered or native meganucleases; TALE-endonucleases (see, for example, U.S. Pat. Nos. 8,586,363 and 9,181,535); and RNA-guided endonucleases, such as those of the CRISPR/Cas systems (see, for example, U.S. Pat. Nos. 8,697,359 and 8,771,945 and U.S. Pat. Appl. Pub. No. 2014-0068797). One embodiment of the disclosure thus relates to utilizing a nuclease or any associated protein to carry out genome modification. This nuclease could be provided heterologously within donor template DNA for templated-genomic editing or in a separate molecule or vector. A recombinant DNA construct may also comprise a sequence encoding one or more guide RNAs to direct the nuclease to the site within the plant genome to be modified. Further methods for altering or introducing a single genetic locus include, for example, utilizing single-stranded oligonucleotides to introduce base pair modifications in a grape plant genome (see, for example Sauer et al., Plant Physiol, 170(4):1917-1928, 2016).

Methods for site-directed alteration or introduction of a single genetic locus are well-known in the art and include those that utilize sequence-specific nucleases, such as the aforementioned, or complexes of proteins and guide-RNA that cut genomic DNA to produce a double-strand break (DSB) or nick at a genetic locus. As is well-understood in the art, during the process of repairing the DSB or nick introduced by the nuclease enzyme, a donor template, transgene, or expression cassette polynucleotide may become integrated into the genome at the site of the DSB or nick. The presence of homology arms in the DNA to be integrated may promote the adoption and targeting of the insertion sequence into the plant genome during the repair process through homologous recombination or non-homologous end joining (NHEJ).

In another embodiment of the present disclosure, genetic transformation may be used to insert a selected transgene into a plant or may, alternatively, be used for the preparation of transgenes which can be introduced by backcrossing. Methods for the transformation of plants that are well-known to those of skill in the art and applicable to many crop species include, but are not limited to, electroporation, microprojectile bombardment, Agrobacterium-mediated transformation, and direct DNA uptake by protoplasts.

To effect transformation by electroporation, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wound tissues in a controlled manner.

An efficient method for delivering transforming DNA segments to plant cells is microprojectile bombardment. In this method, particles are coated with nucleic acids and delivered into cells by a propelling force. Exemplary particles include those comprised of tungsten, platinum, and preferably, gold. For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into plant cells by acceleration is the Biolistics Particle Delivery System, which can be used to propel particles coated with DNA or cells through a screen, such as a stainless steel or Nytex screen, onto a surface covered with target cells. The screen disperses the particles so that they are not delivered to the recipient cells in large aggregates. Microprojectile bombardment techniques are widely applicable, and may be used to transform virtually any plant species.

Agrobacterium-mediated transfer is another widely applicable system for introducing gene loci into plant cells. An advantage of the technique is that DNA can be introduced into whole plant tissues, thereby bypassing the need for regeneration of an intact plant from a protoplast. Modern Agrobacterium transformation vectors are capable of replication in E. coli as well as Agrobacterium, allowing for convenient manipulations (Klee et al., Nat. Biotechnol., 3(7):637-642, 1985). Moreover, recent technological advances in vectors for Agrobacterium-mediated gene transfer have improved the arrangement of genes and restriction sites in the vectors to facilitate the construction of vectors capable of expressing various polypeptide coding genes. The vectors described have convenient multi-linker regions flanked by a promoter and a polyadenylation site for direct expression of inserted polypeptide coding genes. Additionally, Agrobacterium containing both armed and disarmed Ti genes can be used for transformation.

In those plant strains where Agrobacterium-mediated transformation is efficient, it is the method of choice because of the facile and defined nature of the gene locus transfer. The use of Agrobacterium-mediated plant integrating vectors to introduce DNA into plant cells is well known in the art (Fraley et al., Nat. Biotechnol., 3:629-635, 1985; U.S. Pat. No. 5,563,055).

Transformation of plant protoplasts also can be achieved using methods based on calcium phosphate precipitation, polyethylene glycol treatment, electroporation, and combinations of these treatments (see, for example, Potrykus et al., Mol. Gen. Genet., 199:183-188, 1985; Omirulleh et al., Plant Mol. Biol., 21(3):415-428, 1993; Fromm et al., Nature, 312:791-793, 1986; Uchimiya et al., Mol. Gen. Genet., 204:204, 1986; Marcotte et al., Nature, 335:454, 1988). Transformation of plants and expression of foreign genetic elements is exemplified in Choi et al. (Plant Cell Rep., 13:344-348, 1994), and Ellul et al. (Theor. Appl. Genet., 107:462-469, 2003).

### V. Definitions

The following definitions are provided to better define the present invention and to guide those of ordinary skill in the art in the practice of the present invention. Unless otherwise noted, terms are to be understood according to conventional usage by those of ordinary skill in the relevant art.

As used herein, the term “plant” includes plant cells, plant protoplasts, embryos, plant cells of tissue culture from which grape plants can be regenerated, plant calli, plant clumps and plant cells that are intact in plants or parts of plants such as pollen, flowers, seeds, embryos, leaves, stems, and the like. In certain embodiments, the plant part can be a non-regenerable portion of a plant part. As used in this context, a “non-regenerable” portion of a plant part is a portion that cannot be induced to form a whole plant or that cannot be induced to form a whole plant that is capable of sexual and/or asexual reproduction. In certain embodiments, a non-regenerable portion of a plant part is a portion of a berry, seed, leaf, flower, stem, or root. In particular embodiments, the plant or part thereof can be a grape plant, table grape variety, a green table grape plant, a red table grape plant, or interspecific hybrid Vitis plant.

As used herein, a “control” plant, plant seed, embryo, plant part, plant cell, and/or plant genome may also be a plant, plant seed, plant part, embryo, plant cell, and/or plant genome having a similar (but not the same or identical) genetic background to a modified plant, plant seed, plant part, plant cell, and/or plant genome, if deemed sufficiently similar for comparison of the characteristics or traits to be analyzed.

As used herein, the term “population” means a genetically heterogeneous collection of plants that share a common parental derivation.

As used herein, the terms “variety” and “cultivar” mean a group of similar plants that by their genetic pedigrees and performance can be identified from other varieties within the same species.

As used herein, an “allele” refers to one of two or more alternative forms of a genomic sequence at a given locus on a chromosome.

A “quantitative trait locus” (QTL) is a chromosomal location that encodes for at least a first allele that affects the expressivity of a phenotype.

As used herein, a “marker” means a detectable characteristic that can be used to discriminate between organisms. Examples of such characteristics include, but are not limited to, genetic markers, biochemical markers, metabolites, morphological characteristics, and agronomic characteristics.

As used herein, the term “phenotype” means the detectable characteristics of a cell or organism that can be influenced by gene expression.

As used herein, the term “genotype” means the specific allelic makeup of a plant.

As used herein, “elite” or “cultivated” variety means any variety that has resulted from breeding and selection for superior agronomic performance. An “elite plant” refers to a plant belonging to an elite variety. Numerous elite varieties are available and known to those of skill in the art of grape breeding. An “elite population” is an assortment of elite individuals or varieties that can be used to represent the state of the art in terms of agronomically superior genotypes of a given crop species, such as grape. Similarly, an “elite germplasm” or elite strain of germplasm is an agronomically superior germplasm.

As used herein, the term “introgressed,” when used in reference to a genetic locus, refers to a genetic locus that has been introduced into a new genetic background, such as through backcrossing. Introgression of a genetic locus can be achieved through plant breeding methods and/or by molecular genetic methods. Such molecular genetic methods include, but are not limited to, various plant transformation techniques and/or methods that provide for homologous recombination, non-homologous recombination, site-specific recombination, and/or genomic modifications that provide for locus substitution or locus conversion.

As used herein, the terms “recombinant” or “recombined” in the context of a chromosomal segment refer to recombinant DNA sequences comprising one or more genetic loci in a configuration in which they are not found in nature, for example as a result of a recombination event between homologous chromosomes during meiosis.

As used herein, the term “linked,” when used in the context of nucleic acid markers and/or genomic regions, means that the markers and/or genomic regions are located on the same linkage group or chromosome such that they tend to segregate together at meiosis.

As used herein, “tolerance locus” means a locus associated with tolerance to, or reduction in berry browning. For instance, a tolerance locus according to the present disclosure may, in one embodiment, control tolerance or susceptibility to berry browning.

As used herein, “tolerance” or “improved tolerance” in a plant refers to the ability of the plant to perform well, for example by maintaining normal pigmentation, under browning conditions. Tolerance may also refer to the ability of a plant to maintain a plant vigor phenotype under disease conditions. Tolerance is a relative term, indicating that a “tolerant” plant is more able to maintain performance compared to a different (less tolerant) plant (e.g. a different plant variety) grown in similar disease conditions. One of skill will appreciate that plant tolerance to disease conditions varies widely, and can represent a spectrum of more-tolerant or less-tolerant phenotypes. However, by simple observation, one of skill can generally determine the relative tolerance of different plants, plant varieties, or plant families under disease conditions, and furthermore, will also recognize the phenotypic gradations of “tolerance.”

As used herein “resistance” or “improved resistance” in a plant to unfavorable conditions is an indication that the plant is more able to reduce an undesirable phenotype than a non-resistant or less resistant plant. Resistance is a relative term, indicating that a “resistant” plant is more able to reduce berry browning compared to a different (less resistant) plant (e.g., a different plant variety) grown and/or harvested in similar conditions. One of skill will appreciate that plant resistance to berry browning varies widely, and can represent a spectrum of more-resistant or less-resistant phenotypes. However, by simple observation, one of skill can generally determine the relative resistance of different plants, plant varieties, or plant families under unfavorable conditions, and furthermore, will also recognize the phenotypic gradations of “resistant.”

As used herein “reduced” or “reduction” in the context of berry browning is an indication that the plant exhibits a delay in onset of berry browning, a decrease in the extent of berry browning, a decrease in one or more specific types of browning (e.g. external, internal, low temperature, chemical, physical, and pathogenic browning) or any combination thereof. Reduction is a relative term, indicating that a plant comprising “reduced berry browning” or “reduced oxidative browning” exhibits less berry browning compared to a different plant (e.g., a different plant variety) grown and/or harvested in similar conditions. One of skill will appreciate that reduced berry browning varies widely, and can represent a spectrum of more-reduced or less-reduced phenotypes. However, by simple observation, one of skill can generally determine the relative reduction in berry browning of different plants, plant varieties, or plant families under unfavorable conditions, and furthermore, will also recognize the phenotypic gradations of “reduced” berry browning, which can be determined by both qualitative and quantitative means.

As used herein, “polymorphism” means the presence of one or more variations of a nucleic acid sequence at one or more loci in a population of one or more individuals. The variation may comprise but is not limited to one or more base changes, the insertion of one or more nucleotides or the deletion of one or more nucleotides. A polymorphism may arise from random processes in nucleic acid replication, through mutagenesis, as a result of mobile genomic elements, from copy number variation and during the process of meiosis, such as unequal crossing over, genome duplication and chromosome breaks and fusions. The variation can be commonly found or may exist at low frequency within a population, the former having greater utility in general plant breeding and the latter may be associated with rare but important phenotypic variation. Useful polymorphisms may include single nucleotide polymorphisms (SNPs), insertions or deletions in DNA sequence (Indels), simple sequence repeats of DNA sequence (SSRs) a restriction fragment length polymorphism, and a tag SNP. A genetic marker, a gene, a DNA-derived sequence, a haplotype, a RNA-derived sequence, a promoter, a 5′ untranslated region of a gene, a 3′ untranslated region of a gene, microRNA, siRNA, a QTL, a satellite marker, a transgene, mRNA, ds mRNA, a transcriptional profile, and a methylation pattern may comprise polymorphisms.

As used herein, “resistance allele(s)” means the nucleic acid sequence(s) associated with tolerance to, resistance to, or reduction in berry browning.

The term “about” is used to indicate that a value includes the standard deviation of error for the device or method being employed to determine the value. The use of the term “or” in the claims is used to mean “and/or” unless explicitly indicated to refer to alternatives only or the alternatives are mutually exclusive, although the disclosure supports a definition that refers to only alternatives and to “and/or.” When used in conjunction with the word “comprising” or other open language in the claims, the words “a” and “an” denote “one or more,” unless specifically noted. The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps. Similarly, any plant that “comprises,” “has” or “includes” one or more traits is not limited to possessing only those one or more traits and covers other unlisted traits.

### VI. Deposit Information

A deposit of representative sample of plant tissue of Vitis vinifera line ‘04009-039-230’, which comprises the chromosomal segments on chromosomes 9 and 18 as described herein was made with the the Provasoli-Guillard National Center for Marine Algae and Microbiota (NCMA), 60 Bigelow Drive, East Boothbay, Me., 04544 USA. The deposit was assigned NCMA Accession No. 202110015. The date of deposit of the representative sample of plant tissue with the NCMA was Oct. 6, 2021. The deposit has been accepted under the Budapest Treaty and will be maintained in the NCMA depository for a period of 30 years, or 5 years after the most recent request, or for the enforceable life of the patent, whichever is longer, and will be replaced if necessary during that period. Upon issuance, all restrictions on the availability to the public of the deposit will be irrevocably removed consistent with all of the requirements of the Budapest Treaty and 37 C.F.R. §§ 1.801-1.809. Applicant does not waive any infringement of rights granted under this patent or under the Plant Variety Protection Act (7 USC 2321 et seq.).

### EXAMPLES

### Example 1. Selection and Scoring of Table Grape Selections for Genome Wide Association Studies

As described herein, Genome Wide Association Studies (GWAS) were conducted in order to map QTLs by associating a genetic variant with a phenotypic variation. Approximately 76 green seedless selections were obtained for the GWAS panel. The plants were unreleased numbered selections from the International Fruit Genetics table grape breeding program.

In brief, fruit samples were harvested at 18° Brix, and 20 berries from each selection were randomly chosen for evaluation. Individual berries were removed from the rachis and visual scoring of browning was conducted. The percentage of sample that showed browning after 24 hours at room temperature was recorded.

### Example 2. Genome Wide Association Studies of Table Grape Panel

Young leaf samples were harvested from each selection and submitted to an external laboratory for genotyping. A modified version of genotypoing-by-sequencing was conducted (Ott et al. 2017) using the restriction enzyme Bsp1286I. Samples were barcoded and sequenced using an Illumina HiSeq X instrument. Raw sequence data was de-barcoded and reads were assigned to their corresponding samples before quality trimming. Quality trimmed reads were aligned to the 12X.0 version of the PN40024 V. vinifera reference genome (Jaillon et al. 2007). SNPs were called on the aligned reads and only those present in 95% of samples were utilized. A set of 163,000 genome-wide SNPs were included in the subsequent GWAS analysis.

Genome wide association analysis was conducted using the mixed-linear model MLMM method (Segura et al. 2012) implemented in GAPIT v. 3.0 (Wang and Zhang 2021) that accounts for population structure and kinship within the population. The final genome-wide significance thresholds were calculated by GEC (Li et al. 2012), which applies a Bonferroni multiple test correction.

Significant loci associated with decreased berry browning were detected on chromosomes 9 (snp9_17600454) and 18 (snp18_27007362) when using an additive genetic model; and on chromosome 5 (snp5_17373984) when using a model allowing for dominance. Additional markers defining the QTL intervals on each chromosome were also identified and are provided in Table 1 below.

TABLE 1
List of markers and favorable alleles at each marker
for tracking berry browning reduction QTLs.

| | | Public | | | | | Marker |
|---|---|---|---|---|---|---|---|
| Marker | | position of | Marker | SNP position | SNP | Favorable | sequence |
| name | Chr. | SNP (bp) | size (bp) | in marker (bp) | change | allele | (SEQ ID NO) |
| M1 | 5 | 17,371,597 | 101 | 51 | [G/A] | G | 1 |
| M2 | 5 | 17,371,709 | 101 | 51 | [T/C] | T | 2 |
| M3 | 5 | 17,373,984 | 101 | 51 | [G/C] | G | 3 |
| M4 | 5 | 17,373,984 | 101 | 51 | [G/C] | C | 4 |
| M5 | 5 | 17,374,133 | 101 | 51 | [G/C] | C | 5 |
| M6 | 5 | 17,401,109 | 101 | 51 | [C/T] | T | 6 |
| M7 | 9 | 17,551,003 | 101 | 51 | [A/C] | A | 7 |
| M8 | 9 | 17,580,578 | 101 | 51 | [C/G] | G | 8 |
| M9 | 9 | 17,600,454 | 101 | 51 | [C/T] | T | 9 |
| M10 | 9 | 17,600,517 | 101 | 51 | [C/T] | C | 10 |
| M11 | 9 | 17,600,713 | 101 | 51 | [A/G] | G | 11 |
| M12 | 18 | 27,001,788 | 101 | 51 | [A/T] | T | 12 |
| M13 | 18 | 27,001,811 | 101 | 51 | [G/A] | G | 13 |
| M14 | 18 | 27,007,362 | 101 | 51 | [A/C] | C | 14 |
| M15 | 18 | 27,007,362 | 101 | 51 | [A/C] | A | 15 |
| M16 | 18 | 27,007,513 | 101 | 51 | [C/A] | C | 16 |
| M17 | 18 | 27,007,611 | 101 | 51 | [A/G] | G | 17 |

### Example 3. Effect of Allele Substitution on Reduction of Berry Browning

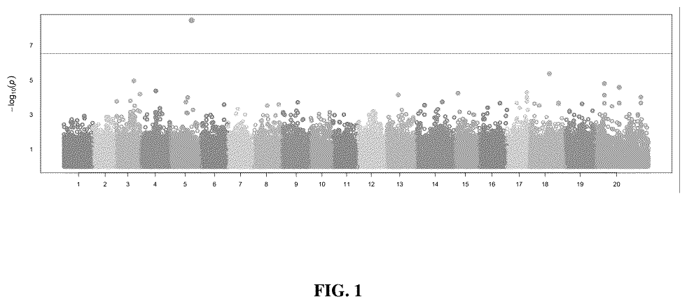

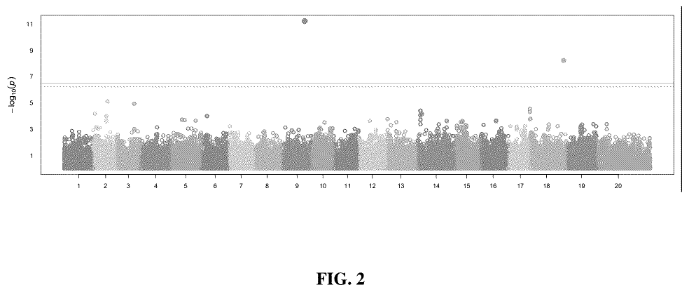

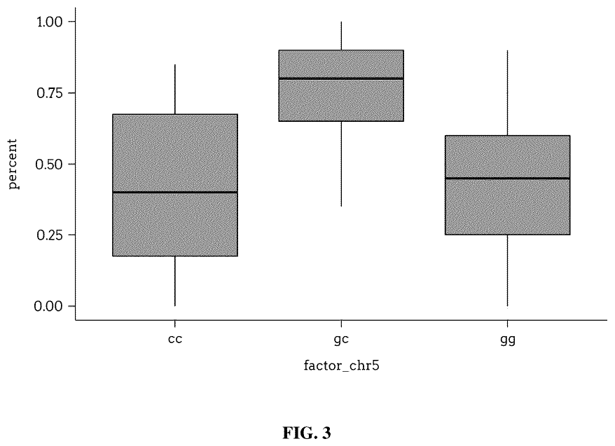

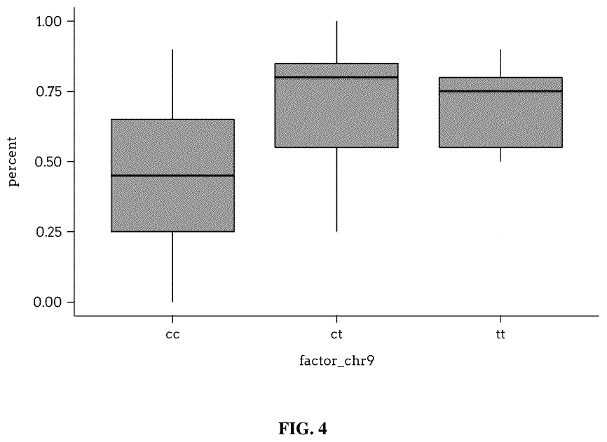

The GWAS identified significant loci on chromosome 5 (FIG. 1); and chromosome 9 and chromosome 18 (FIG. 2). The favorable genotype “GC” on chromosome 5 was associated with an approximately 25% decrease in the percentage of berry browning (FIG. 3). The favorable allele “T” on chromosome 9 was also associated with an approximately 25% decrease in berry browning (FIG. 4). The favorable genotype “AC” on chromosome 18 was associated with an approximately 15% decrease in berry browning (FIG. 3).

### Example 4. Introgression of Berry Browning Reduction Loci

The QTLs on chromosomes 5, 9, and 18 will be further validated using a large panel of Vitis vinifera selections and interspecific hybrids analogous to the panel described in Example 1. The effect of each QTL as well as the combination of QTLs described herein will be validated using phenotypes for reduction of to berry browning and genotypes at the QTLs.

Additionally, the markers disclosed will be used to perform Marker Assisted Selection (MAS) for reducing berry browning. For example, plant breeders will use these molecular markers to create lines resistant to Berry Browning by selecting the favorable allele(s) and genotypes described herein. Stacking the berry browning QTLs on chromosome 5, 9, and 18, will be performed to create lines outperforming lines carrying only one or two of the QTLs for berry browning reduction.

What is claimed is:

1. A Vitis vinifera plant comprising at least a first introgressed allele on a first chromosome selected from the group consisting of chromosomes 5, 9, and 18, and wherein said first introgressed allele confers to said plant a reduction in berry browning compared to a plant not comprising said allele.

2. The Vitis vinifera plant of claim 1, wherein said plant further comprises a second introgressed allele on a second chromosome selected from the group consisting of chromosomes 5, 9, and 18, wherein said second introgressed allele confers to a plant a reduction in berry browning compared to a plant not comprising said allele.

3. The Vitis vinifera plant of claim 2, wherein said plant further comprises a third introgressed allele on a third chromosome selected from the group consisting of chromosomes 5, 9, and 18, wherein said third introgressed allele confers to a plant a reduction in berry browning compared to a plant not comprising said allele.

4. The Vitis vinifera plant of claim 1, wherein said first introgressed allele is flanked in the genome of said plant by:
a) marker locus M1 (SEQ ID NO: 1) and marker locus M6 (SEQ ID NO: 6) on chromosome 5;
b) marker locus M7 (SEQ ID NO: 7) and marker locus M11 (SEQ ID NO: 11) on chromosome 9; or
c) marker locus M12 (SEQ ID NO: 12) and marker locus M17 (SEQ ID NO:17) on chromosome 18.

5. The Vitis vinifera plant of claim 3, wherein a representative deposit of a plant comprising said first, second, and third alleles has been deposited under NCMA Accession No. 202110015.

6. The Vitis vinifera plant of claim 4, wherein said first introgressed allele is located at a position in the genome of said plant flanked by marker locus M1 (SEQ ID NO: 1) and marker locus M6 (SEQ ID NO: 6) on chromosome 5 and wherein said allele comprises the haplotype for said allele found in Vitis vinifera variety ‘Valley Pearl’ or ‘Shingargoon’.

7. The Vitis vinifera plant of claim 1, wherein said plant is homozygous for said first introgressed allele.

8. The Vitis vinifera plant of claim 1, wherein said plant is heterozygous for said first introgressed allele.

9. The Vitis vinifera plant of claim 2, wherein said plant is homozygous for said second introgressed allele.

10. The Vitis vinifera plant of claim 2, wherein said plant is heterozygous for said second introgressed allele.

11. The Vitis vinifera plant of claim 3, wherein said plant is homozygous for said third introgressed allele.

12. The Vitis vinifera plant of claim 3, wherein said plant is heterozygous for said third introgressed allele.

13. The Vitis vinifera plant of claim 8, wherein said first introgressed allele is on chromosome 5 or chromosome 18, and wherein said plant exhibits a reduction in berry browning compared to a plant that homozygous for said first allele.

14. A plant part of the Vitis vinifera plant of claim 1.

15. The plant part of claim 14, wherein the plant part is a cell, a seed, a leaf, a stem, a root, a flower, a berry, a stalk, a root tip, an ovule, an embryo, or pollen.

16. A method for producing a Vitis vinifera plant with reduced oxidative browning comprising:
a) crossing the plant of claim 1 with itself or with a Vitis vinifera plant of a different genotype to produce one or more progeny plants; and
b) selecting a progeny plant comprising said allele that confers reduced oxidative browning.

17. The method of claim 16, wherein selecting said progeny plant comprises detecting a marker locus genetically linked to said reduced oxidative browning allele.

18. The method of claim 17, wherein selecting said progeny plant comprises detecting a marker locus within or genetically linked to a chromosomal segment flanked in the genome of said plant by:
a) marker locus M1 (SEQ ID NO: 1) and marker locus M6 (SEQ ID NO: 6) on chromosome 5;
b) marker locus M7 (SEQ ID NO: 7) and marker locus M11 (SEQ ID NO: 11) on chromosome 9; or
c) marker locus M12 (SEQ ID NO: 12) and marker locus M17 (SEQ ID NO:17) on chromosome 18.

19. The method of claim 17, wherein selecting a progeny plant comprises detecting nucleic acids comprising a marker locus selected from the group consisting of marker locus M1 (SEQ ID NO: 1), marker locus M2 (SEQ ID NO: 2), marker locus M3 (SEQ ID NO: 3), marker locus M4 (SEQ ID NO: 4), marker locus M5 (SEQ ID NO: 5), marker locus M6 (SEQ ID NO: 6), marker locus M7 (SEQ ID NO: 7), marker locus M8 (SEQ ID NO: 8), marker locus M9 (SEQ ID NO: 9), marker locus M10 (SEQ ID NO: 10), marker locus M11 (SEQ ID NO: 11), marker locus M12 (SEQ ID NO: 12), marker locus M13 (SEQ ID NO: 13), marker locus M14 (SEQ ID NO: 14), marker locus M15 (SEQ ID NO: 15), marker locus M16 (SEQ ID NO: 16), and marker locus M17 (SEQ ID NO: 17), or a marker locus located within 10 cM thereof.

20. The method of claim 19, wherein selecting a progeny plant comprises detecting nucleic acids comprising marker locus M3 (SEQ ID NO: 3) and marker locus M4 (SEQ ID NO: 4) or marker locus M14 (SEQ ID NO: 14) and marker locus M15 (SEQ ID NO: 15).

21. The method of claim 18, wherein selecting said progeny plant comprises identifying a genetic marker within or genetically linked to a genomic region between marker locus M7 (SEQ ID NO: 7) and marker locus M11 (SEQ ID NO: 11) on chromosome 9.

22. The method of claim 21, wherein selecting said progeny plant further comprises detecting at least one polymorphism at a locus selected from the group consisting of marker locus marker locus M8 (SEQ ID NO: 8), M9 (SEQ ID NO: 9), and marker locus M10 (SEQ ID NO: 10).

23. The method of claim 16, wherein the progeny plant is an F₂-F₆ progeny plant.

24. A method of producing a plant of a table grape variety exhibiting reduced berry browning, comprising introgressing into a plant a reduced oxidative browning allele, wherein said reduced oxidative browning allele is defined as located in a genomic region between:
a) marker locus M1 (SEQ ID NO: 1) and marker locus M6 (SEQ ID NO: 6) on chromosome 5;
b) marker locus M7 (SEQ ID NO: 7) and marker locus M11 (SEQ ID NO: 11) on chromosome 9; or
c) marker locus M12 (SEQ ID NO: 12) and marker locus M17 (SEQ ID NO:17) on chromosome 18.

25. The method of claim 24, wherein said introgressing comprises backcrossing or marker-assisted selection.

26. The method of claim 24, wherein said introgressing comprises detecting nucleic acids comprising marker locus M1 (SEQ ID NO: 1), marker locus M2 (SEQ ID NO: 2), marker locus M3 (SEQ ID NO: 3), marker locus M4 (SEQ ID NO: 4), marker locus M5 (SEQ ID NO: 5), marker locus M6 (SEQ ID NO: 6), marker locus M7 (SEQ ID NO: 7), marker locus M8 (SEQ ID NO: 8), marker locus M9 (SEQ ID NO: 9), marker locus M10 (SEQ ID NO: 10), marker locus M11 (SEQ ID NO: 11), marker locus M12 (SEQ ID NO: 12), marker locus M13 (SEQ ID NO: 13), marker locus M14 (SEQ ID NO: 14), marker locus M15 (SEQ ID NO: 15), marker locus M16 (SEQ ID NO: 16), or marker locus M17 (SEQ ID NO: 17).

27. A method of selecting a plant of a table grape variety exhibiting reduced berry browning, comprising:
a) obtaining a population of progeny plants having at least one parent exhibiting reduced berry browning;
b) screening said population with at least one nucleic acid marker to detect a locus associated with an allele that confers reduced oxidative browning; and
c) selecting from said population one or more progeny plants based on the presence of said locus, wherein said locus is within or genetically linked to a chromosomal segment flanked in the genome of said plant by:
i) marker locus M1 (SEQ ID NO: 1) and marker locus M6 (SEQ ID NO: 6) on chromosome 5;
ii) marker locus M7 (SEQ ID NO: 7) and marker locus M11 (SEQ ID NO: 11) on chromosome 9; or
iii) marker locus M12 (SEQ ID NO: 12) and marker locus M17 (SEQ ID NO:17) on chromosome 18.

28. The method of claim 27, wherein selecting said progeny plants comprises detecting nucleic acids comprising a marker locus selected from the group consisting of marker locus M1 (SEQ ID NO: 1), marker locus M2 (SEQ ID NO: 2), marker locus M3 (SEQ ID NO: 3), marker locus M4 (SEQ ID NO: 4), marker locus M5 (SEQ ID NO: 5), marker locus M6 (SEQ ID NO: 6), marker locus M7 (SEQ ID NO: 7), marker locus M8 (SEQ ID NO: 8), marker locus M9 (SEQ ID NO: 9), marker locus M10 (SEQ ID NO: 10), marker locus M11 (SEQ ID NO: 11), marker locus M12 (SEQ ID NO: 12), marker locus M13 (SEQ ID NO: 13), marker locus M14 (SEQ ID NO: 14), marker locus M15 (SEQ ID NO: 15), marker locus M16 (SEQ ID NO: 16), and marker locus M17 (SEQ ID NO: 17), or a marker locus located within 10 cM thereof.

29. The method of claim 27, wherein said progeny plant is an F₂-F₆ progeny plant.

30. The method of claim 27, wherein screening said population comprises PCR, single strand conformational polymorphism analysis, denaturing gradient gel electrophoresis, cleavage fragment length polymorphism analysis, TAQMAN assay, and/or DNA sequencing.

31. A method for identifying a Vitis vinifera plant comprising a reduced oxidative browning allele:
a) obtaining nucleic acids from at least a first Vitis vinifera plant; and
b) identifying in said nucleic acids the presence of at least a first genetic marker indicative of the presence of a chromosomal segment flanked in the genome of said plant by:
i) marker locus M1 (SEQ ID NO: 1) and marker locus M6 (SEQ ID NO: 6) on chromosome 5;
ii) marker locus M7 (SEQ ID NO: 7) and marker locus M11 (SEQ ID NO: 11) on chromosome 9; or
iii) marker locus M12 (SEQ ID NO: 12) and marker locus M17 (SEQ ID NO:17) on chromosome 18,
wherein said reduced oxidative browning allele confers to said plant reduced berry browning compared to a plant not comprising said allele.

32. The method of claim 31, wherein said identifying comprises detecting a marker genetically linked to:
a) marker locus M2 (SEQ ID NO: 2), marker locus M3 (SEQ ID NO: 3), marker locus M4 (SEQ ID NO: 4), and marker locus M5 (SEQ ID NO: 5);
b) marker locus M8 (SEQ ID NO: 8), marker locus M9 (SEQ ID NO: 9), and marker locus M10 (SEQ ID NO: 10); or
c) marker locus M13 (SEQ ID NO: 13), marker locus M14 (SEQ ID NO: 14), marker locus M15 (SEQ ID NO: 15), and marker locus M16 (SEQ ID NO: 16).

33. The method of claim 31, wherein the Vitis vinifera plant is a plant embryo.

34. A seed that produces the Vitis vinifera plant of claim 1.

35. An embryo that produces the Vitis vinifera plant of claim 1.

36. A Vitis vinifera plant obtainable by the method of claim 16, wherein said plant exhibits reduced oxidative browning.

37. A Vitis vinifera plant obtainable by the method of claim 24, wherein said plant exhibiting reduced berry browning.

38. An interspecific hybrid Vitis plant produced from a cross between a Vitis vinifera plant and a plant of a distinct Vitis species, or a progeny thereof, comprising at least a first introgressed allele on a first chromosome selected from the group consisting of chromosomes 5, 9, and 18, and wherein said first introgressed allele confers to said plant a reduction in berry browning compared to a plant not comprising said allele.

39. An embryo that produces the interspecific hybrid Vitis plant of claim 38.

40. A progeny plant of the plant of claim 1, wherein the plant comprises said first introgressed allele.


SEQUENCE LISTING (17 sequences)

SEQ ID NO: 1 - DNA, 101 bp, Vitis vinifera
  source 1..101: mol_type = genomic DNA; note = Vitis vinifera L.
attaagactttcaagtgagacagaaaagaagtttcccatcttcatttgccgatgatacaattctttcctcaattttacctatcaaaaaaatataaaaaaat

SEQ ID NO: 2 - DNA, 101 bp, Vitis vinifera
  source 1..101: mol_type = genomic DNA; note = Vitis vinifera L.
ttccttcttcatggaaggagaataaggaaccacatacttgttagaggttattgaatttataacacactttagaattaatatagccaagagtgcacttcttg

SEQ ID NO: 3 - DNA, 101 bp, Vitis vinifera
  source 1..101: mol_type = genomic DNA; note = Vitis vinifera L.
aagggttttgctttgttagtggggcacaagaaggtaaacatcaatgacatcctacagttgaaaagatcttacaaattccttagtcctcattggtgcatctt

SEQ ID NO: 4 - DNA, 101 bp, Vitis vinifera
  source 1..101: mol_type = genomic DNA; note = Vitis vinifera L.
aagggttttgctttgttagtggggcacaagaaggtaaacatcaatgacatgctacagttgaaaagatcttacaaattccttagtcctcattggtgcatctt

SEQ ID NO: 5 - DNA, 101 bp, Vitis vinifera
  source 1..101: mol_type = genomic DNA; note = Vitis vinifera L.
tgaatttataacacactttagaattaatatagccaagagtgcacttcttgggattctaccctagagaatgtgacatgatgaccatctcatttagaggtttg

SEQ ID NO: 6 - DNA, 101 bp, Vitis vinifera
  source 1..101: mol_type = genomic DNA; note = Vitis vinifera L.
tagaccaaatcaaacaaaacattagttctcaccatcatgatatgccaggtcgctattatgcttatgcaactggcagcatgttgttatcaagcaaagcagat

SEQ ID NO: 7 - DNA, 101 bp, Vitis vinifera
  source 1..101: mol_type = genomic DNA; note = Vitis vinifera L.
ttaaaattttgatttcattaaaactcgagttttatcgattgtaccttaggaaaaatgtttcaaaattggcatattaattcacctaaagactttgactaagt

SEQ ID NO: 8 - DNA, 101 bp, Vitis vinifera
  source 1..101: mol_type = genomic DNA; note = Vitis vinifera L.
aagtatgctagtcaaacttagggtgctgaatgcgaacaagcttccatgagcatctaaaagagcaccttcccttggccttctcctcttgaaactctgtcata

SEQ ID NO: 9 - DNA, 101 bp, Vitis vinifera
  source 1..101: mol_type = genomic DNA; note = Vitis vinifera L.
ggttgatttcagattgtgattgggggcccaagcccatacaataacacaatccaggtccatgcccaacaatattattttaaaacgggccataagggcctaaa

SEQ ID NO: 10 - DNA, 101 bp, Vitis vinifera
  source 1..101: mol_type = genomic DNA; note = Vitis vinifera L.
caacaatattattttaaaacgggccataagggcctaaactggccattttccttattaagcccatgacccaacaatttactaaaactagggattaaaaaggc

SEQ ID NO: 11 - DNA, 101 bp, Vitis vinifera
  source 1..101: mol_type = genomic DNA; note = Vitis vinifera L.
attgccaatgctccacagtatgaccttggttgcccttcaagtttagattaagcttcacttaggtgcactttaggttagactttagttaggcccacctagtc

SEQ ID NO: 12 - DNA, 101 bp, Vitis vinifera
  source 1..101: mol_type = genomic DNA; note = Vitis vinifera L.
cgaaaccctcaaaaaagttatgaatgtgaaaacaagccttttacatttctataatgaaaatgataaacatcagggtaaaaatagaatcaaagtgaatcaat

SEQ ID NO: 13 - DNA, 101 bp, Vitis vinifera
  source 1..101: mol_type = genomic DNA; note = Vitis vinifera L.
atgtgaaaacaagccttttacatttctataatgaaaatgataaacatcagggtaaaaatagaatcaaagtgaatcaatcaaaggataaagtatcaggccca

SEQ ID NO: 14 - DNA, 101 bp, Vitis vinifera
  source 1..101: mol_type = genomic DNA; note = Vitis vinifera L.
attttcatactcaatacctaaaatcaaataaagcaaatcacaaaccaatcaaagcccccctagaagaaggatatagaatattagtagtagctagtgaagaa

SEQ ID NO: 15 - DNA, 101 bp, Vitis vinifera
  source 1..101: mol_type = genomic DNA; note = Vitis vinifera L.
attttcatactcaatacctaaaatcaaataaagcaaatcacaaaccaatccaagcccccctagaagaaggatatagaatattagtagtagctagtgaagaa

SEQ ID NO: 16 - DNA, 101 bp, Vitis vinifera
  source 1..101: mol_type = genomic DNA; note = Vitis vinifera L.
atctgcattaatgctcttcacttcattacataaaattgtgtgccccttcacagtctgtgacatgaagattcaaagtcgatgacaaattaatgaagatgaac

SEQ ID NO: 17 - DNA, 101 bp, Vitis vinifera
  source 1..101: mol_type = genomic DNA; note = Vitis vinifera L.
aactttcttatgacacaaaatgtccaggctctgattggttgctaagaaaaagcaggaaaagagaagtaaaattctgaatcttcaattttgcattactctgc